(19) United States
(12) Patent Application Publication
(10) Pub. No.: US 2026/0345309 A1
(43) Pub. Date: Nov. 5, 2026

(54) METHODS AND SYSTEMS FOR IMPROVED BIOCOMPATIBLE MEMBRANES FOR IMPLANTABLE DEVICES

(71) Applicant: PROCYON TECHNOLOGIES LLC, Tucson, AZ (US)

(72) Inventors: Klearchos PAPAS, Tucson, AZ (US); Robert C. JOHNSON, Estes Park, CO (US)

(73) Assignee: ARIZONA BOARD OF REGENTS ON BEHALF OF THE UNIVERSITY OF ARIZONA, Tucson, AZ (US)

(21) Appl. No.: 19/486,770

(22) PCT Filed: May 21, 2024
(86) PCT No.: PCT/US2024/030321

§ 371 Date: Nov. 21, 2025

Related U.S. Application Data
(60) Provisional application No. 63/468,188, filed on May 22, 2023.

(51) Int. Cl. A61F 2/02; A61F 2/00; A61M 31/00

(52) U.S. Cl. CPC A61F 2/022; A61M 31/002; A61F 2002/0081; A61F 2002/0086; A61F 2210/0076

(57) ABSTRACT

Improved membranes for various devices including encapsulation devices and delivery devices are provided. Membranes of the present disclosure provide various combinations and layers of structures and features shown and described herein. In some embodiments, a spacing is provided between certain layers to enhance vascularization of the device. The present disclosure further provides devices for delivery of drugs, agent, and for housing cells.

### CROSS-REFERENCE TO RELATED APPLICATIONS

This International Application claims the benefit of priority of United States Provisional Patent Application Ser. No. 63/468,188, filed May 22, 2023, the entire disclosure of which is hereby incorporated by reference.

### FIELD

Embodiments of the present disclosure relate to implantable medical devices including but not limited to cell encapsulation devices and therapeutic agent delivery devices. In some embodiments, implantable encapsulation devices are provided that are operable to house cells, tissue, and/or therapeutic agents and deliver therapeutic effects to a host or recipient of the device. Membranes, layers, and components of implantable devices that are useful in such devices and other applications are disclosed.

### BACKGROUND

The number of patients suffering from Type I and Type II diabetes is estimated to affect about 4.6% of the world's population. Pancreas transplantation and islet transplantation are known methods for treating diabetes. However, pancreas and islet transplantation into diabetic patients is limited to a small percent of patients who might benefit from either procedure due to the lack of available human pancreata or pancreatic islets. With the recent development of insulin secreting cells derived from human stem cells, there is a possibility of treating patients with insulin dependent diabetes through transplantation. However, such cells would be subject to rejection by the immune system of the recipient patient unless immunosuppressive drugs were administered to the patient for the rest of their life. Alternatively, insulin secreting cells could be provided with an immuno-isolating implantable device and placed in the diabetic patient to act as an insulin delivery source.

Since the islet transplantation protocol was established, clinical islet transplantation has been regarded as a treatment method for treating type 1 diabetics. However, the low engraftment success of transplanted islet cells remains a major cause of failure of long-term blood sugar regulation. Upon implantation, it is necessary for islet cells to be successfully engrafted through revascularization and blood flow regulation within a few days after transplantation. However, transplanted islet cells are exposed to a state with low vascular density and insufficient oxygen conditions, making it difficult to achieve normal engraftment of islet cells and the ability to achieve regulated insulin secretion in the patient.

Currently, there are limited means and materials to effectively implement live cell containing immuno-isolation devices in vivo. Limitations associated with supply of adequate oxygen levels to encapsulated cells, sufficient nutrient levels to the encapsulated cells, insufficient vascularization of the implanted device and immune response to the implant, remain barriers to use of cell-containing implantable devices.

Known subcutaneous delivery systems for insulin and other therapeutic agents include cannula-based injection systems. While various transdermal catheter systems are known in the medical industry, such known systems provide for enhanced risk of infection at the implantation site particularly in applications and systems where the catheter is to be integrated into a patient for a long period of time.

### SUMMARY

Embodiments of the present disclosure provide improved membranes that are operable for use with, but are not limited to, implantable medical devices such as implantable encapsulation devices, drug delivery devices, indwelling catheters and the like. It should be recognized that various novel concepts and membranes as provided herein are not limited to any particular intended use and are contemplated as being useful in various situations where vascularization is encouraged or desired and/or where cells are to be housed or managed.

It is an object of the present disclosure to provide devices with improved vascularization capacity. In some embodiments, devices are provided that comprise enhanced surface area for vascularization of an implant, and wherein a ratio of surface area to volume is maximized for promoting vascularization and ultimately improving the efficacy of the device.

In some embodiments, membranes are provided that comprise a plurality of layers. It is further contemplated that adjacent layers of various membranes of the present disclosure are spaced apart as shown and described herein to promote vascularization in one more regions of the membrane.

Membranes of the present disclosure are contemplated as comprising various materials, including those deemed appropriate by a person skilled in the art for an implantable medical device. For example, membranes of the present disclosure are contemplated as being prepared from a polymeric material. In such embodiments, the single layer gradient membrane is prepared from such polymeric materials as: polysulfone, polyarylethersulfone (PAES), polyethersulfone (PES), cellulose ester (cellulose acetate, cellulose triacetate, cellulose nitrate), nanocellulose, regenerated cellulose (RC), silicone, polyamide (nylon), polyimide, polyamide imide, polyamide urea, polycarbonate, ceramic, titanium oxide, aluminum oxide, silicon, zeolite (alumosilicate), polyarylonitrile (PAN), polyethylene (PE), low density polyethylene (LDPE), polypropylene (PP), polytetrafluoroethylene (PTFE), polyvinylidene fluoride (PVDF), polyvinylchloride (PVC), polypiperazine amide, polyethylene terephthalate (PET), polycarbonate (PC), polyurethane, and any complex or mixtures thereof. In particular embodiments, a single layer gradient membrane comprises of a polymeric material comprising polytetrafluoroethylene (PTFE). In certain preferred embodiments, PTFE is provided for at least a vascularizing layer of devices of the present disclosure. Additional materials are contemplated as being provided in membranes and implants of the present disclosure in addition to or in lieu of PTFE.

In various embodiments, it is contemplated that one or more membranes or layers of devices of the present disclosure comprise biodegradable features. For example, in some embodiments, vascularization membranes as disclosed herein are contemplated comprising a biodegradable feature such as disclosed and described in European Patent EP3413941 to Greenwood which is hereby incorporated by reference in its entirety. Additionally, biocompatible or biodegradable polymers such as those shown and described in WO/2008/014561 to Gunatillake et al., WO/2009/043099 to Moore et al., WO/2005/089778 to Adhikari et al., WO/2004/009227 to Adhikari et al., and WO/2005/085312 to Adhikari et al. are contemplated for use in embodiments and devices of the present disclosure and are all hereby incorporated by reference in their entireties.

In some embodiments, a gradient membrane is provided that comprises an electrospun polymeric membrane, such as an electrospun PTFE membrane that is applied directly to a surface, such as a surface of an implantable medical device or a catheter tube. Implantable medical devices of the present disclosure are contemplated as comprising an internal chamber of live cells. A protective layer/film for an internal chamber of an implantable medical device is provided in which live cells may be contained, and the single layer gradient membrane is capable of protecting the cells from immune attack, while simultaneously permitting nutrient flow/oxygen to contained live cells, owing to the appropriate gradient pore size provided by the single layer gradient membrane. Single layer gradient membranes of the present disclosure also provide for a slightly larger pore size within the membrane region extending to the other surface (e.g., outer surface) of the single layer membrane, thus providing a surface suitable for vascularizing the outer surface of an implantable medical device or an indwelling catheter in a host.

In various embodiments, implantable medical devices are provided that comprise at least one surface upon which a single layer membrane material having a gradient structure is applied. The surface is contemplated as comprising the surface of a catheter or an implantable medical device, such as an implantable device that has a lumen comprising living cells (e.g. stem cells).

In one embodiment, membranes are provided that reduce overall fibrosis and comprises pores having a size of about 0.1 to about 100 micron (or, from about 0.1 or about 5 micron to about 15 micron). In some embodiments, an implantable medical device is provided that comprises a lumen comprising living cells. The single layer gradient membrane comprises a pore size that does not interfere with the passage of molecules (such as insulin produced by contained islet cells) out of a lumen chamber, and out of the implantable medical device into the body. In this regard, the membrane is sufficiently thin so as to allow rapid diffusion of molecules out of the implantable medical device. As another example, a single layer gradient membrane is provided on some surfaces of a component of a multi-component implantable medical device and not on other surfaces.

In certain embodiments, systems are provided that comprise a surface having a membrane, such as a membrane comprising a polymeric material. By way of example, the polymeric material is contemplated as comprising PTFE, where the PTFE membrane comprises a gradient of pore sizes. This single layer PTFE gradient membrane is provided to the external surface of the implantable medical device system. The outer side (host vasculature inter-facing) of the PTFE gradient membrane enables cellular ingress (greater than 1 micron to about 15 microns), and the PTFE gradient membrane titrates down in relative pore size to an appropriate size that would prohibit cellular ingress (about 0.1 micron to about 1 micron) into the cell-containing inner chamber of the implantable medical device. The pore size of the PTFE gradient membrane renders the implantable medical device immuno-isolating for the implanted cells.

In further embodiments, implant systems comprise one or more surfaces with an electrospun PTFE gradient membrane combining immunoisolation and vascularization features as described above. An electrospun PTFE multielement layer comprises relatively larger fibers, of a size sufficient to inhibit fibroblast layer formation. This feature may take the form of a final, outer gradient layer comprising multiple strands to form thick fibers of about 25 to about 200 micron in diameter. With such larger fibers randomly oriented on the outer surface of the gradient membrane, the layer serves as a surface to inhibit fibroblasts from forming a fused fibrotic layer.

In various embodiments, the present disclosure provides implantable devices having a number of improved characteristics and features. In some embodiments, an implantable device is provided that possesses a unique configuration that facilitates a maximization of surface area available for vascularization by a host animal. As used herein, the term “animal” refers to animals generally as that term is understood in the art and is not limited to humans or any specific animal.

International Application Nos. PCT/US2017/060036 to Papas, PCT/US2017/060034 to Papas, PCT/US2017/060041 to Papas, and PCT/US2017/060043 to Papas relate to encapsulation devices and are each incorporated by reference in their entireties herein for all purposes.

Devices of the present disclosure comprise various materials, including those deemed appropriate by a person skilled in the art for an implantable medical device. For example, membranes of the present disclosure are contemplated as being prepared from a polymeric material. In such embodiments, the single layer gradient membrane is prepared from such polymeric materials as: polysulfone, polyarylethersulfone (PAES), polyethersulfone (PES), cellulose ester (cellulose acetate, cellulose triacetate, cellulose nitrate), nanocellulose, regenerated cellulose (RC), silicone, polyamide (nylon), polyimide, polyamide imide, polyamide urea, polycarbonate, ceramic, titanium oxide, aluminum oxide, silicon, zeolite (alumosilicate), polyarylonitrile (PAN), polyethylene (PE), low density polyethylene (LDPE), polypropylene (PP), polytetrafluoroethylene (PTFE), polyvinylidene fluoride (PVDF), polyvinylchloride (PVC), polypiperazine amide, polyethylene terephthalate (PET), polycarbonate (PC), polyurethane, and any complex or mixtures thereof. In particular embodiments, a single layer gradient membrane comprises a polymeric material comprising polytetrafluoroethylene (PTFE). In certain preferred embodiments, PTFE is provided for at least a vascularizing layer of devices of the present disclosure. Additional materials are contemplated as being provided in membranes and implants of the present disclosure in addition to or in lieu of PTFE.

In one embodiment, a membrane for implantable medical devices is provided that comprises an immunoisolation membrane comprising a porous layer operable to house or contain cells. A vascularization membrane is provided that is operable to enable and/or permit vasculature to grow into the vascularization layer. The device further comprises a mesh layer and the immunoisolation membrane and the vascularization membrane comprise adjacent and spaced apart layers. A separation is provided between the immunoisolation membrane and the vascularization membrane and the separation is at least approximately 50 microns. Vasculature is operable to be provided in the separation.

In one embodiment, a membrane for implantable medical devices is provided that comprises an immunoisolation layer, a vascularization layer operable to enable and/or permit vasculature to grow into the vascularization layer, a non-woven mesh layer, and a woven mesh layer. Two or more of the layers are provided in a stacked arrangement and secured to one another by at least one of sutures and welds.

Various concepts disclosed herein may be provided in combination with one another even if such combination is not specifically depicted or described.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In addition, the materials, methods, and examples are illustrative only and are not intended to be necessarily limiting.

### DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

The accompanying drawings, which are incorporated in and constitute a part of the specification, illustrate embodiments of the disclosure and together with the general description of the disclosure given above and the detailed description of the drawings given below, serve to explain the principles of the disclosure.

FIG. 1 is a cross-sectional elevation view of a membrane according to an embodiment of the present disclosure.

FIG. 2 is a cross-sectional elevation view of a membrane according to an embodiment of the present disclosure.

FIG. 3 is a cross-sectional elevation view of a membrane according to an embodiment of the present disclosure.

FIG. 4 is a cross-sectional elevation view of a membrane according to an embodiment of the present disclosure.

FIG. 5 is a cross-sectional elevation view of a membrane according to an embodiment of the present disclosure.

FIG. 6 is a cross-sectional elevation view of a membrane according to an embodiment of the present disclosure.

FIG. 7 is a cross-sectional elevation view of a membrane according to an embodiment of the present disclosure.

FIG. 8 is a cross-sectional elevation view of a membrane according to an embodiment of the present disclosure.

FIGS. 9A and 9B are histological views of a membrane and tissue according to an embodiment of the present disclosure.

FIGS. 10A and 10B are histological views of a membrane and tissue according to an embodiment of the present disclosure.

FIGS. 11A and 11B are histological views of a membrane and tissue according to an embodiment of the present disclosure.

FIGS. 12A and 12B are histological views of a membrane and tissue according to an embodiment of the present disclosure.

FIGS. 13A, 13B, 13C and 13D are histological views of a membrane and tissue according to an embodiment of the present disclosure.

FIG. 14A is a top view of a delivery device according to an embodiment of the present disclosure.

FIG. 14B is a cross-sectional side elevation view of a device according to an embodiment of the present disclosure.

FIG. 14C is a cross-sectional side elevation view of a device according to an embodiment of the present disclosure.

FIG. 15A is a top plan view of a delivery device according to an embodiment of the present disclosure.

FIG. 15B is a cross-sectional side elevation view of the device according to FIG. 15A.

FIG. 16A is a top plan view of a delivery device according to an embodiment of the present disclosure.

FIG. 16B is a cross-sectional side elevation view of the device according to FIG. 15A.

FIG. 17 is a cross-sectional side view of a multi-layered encapsulation device according to an embodiment of the present disclosure.

FIG. 18 is a cross-sectional side view of a multi-layered encapsulation device according to an embodiment of the present disclosure.

FIG. 19 is an illustration of a subcutaneous system for sensing and therapeutic treatments according to one embodiment of the present disclosure.

FIG. 20 is a cross-sectional elevation view of the embodiment of FIG. 19.

FIG. 21 is an illustration of an implantable sensor system according to an embodiment of the present disclosure.

FIG. 22 is an illustration of an implantable sensor system according to an embodiment of the present disclosure.

The drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Reference to an element by the indefinite article “a” or “an” does not exclude the possibility that more than one element is present, unless the context clearly requires that there be one and only one element. The indefinite article “a” or “an” thus usually means “at least one.” As used herein, the terms “about” or “approximately” mean within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

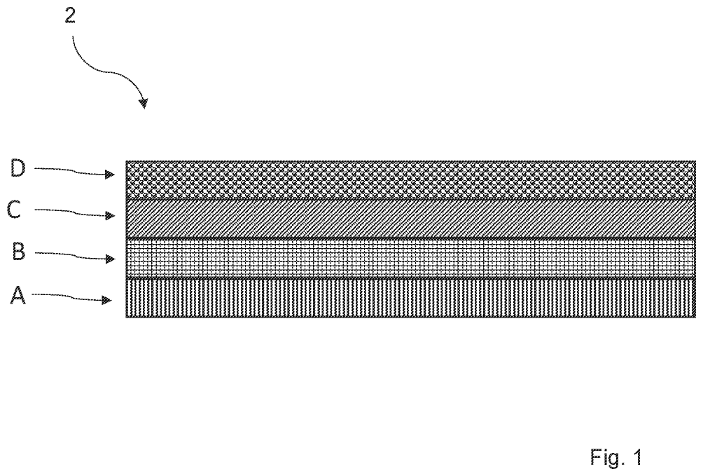

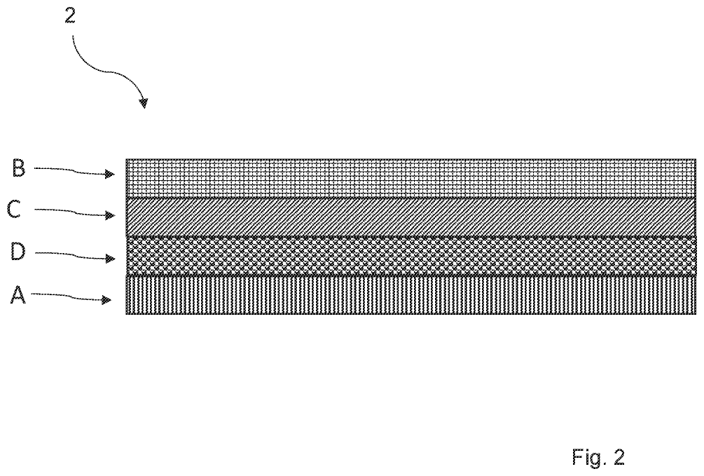

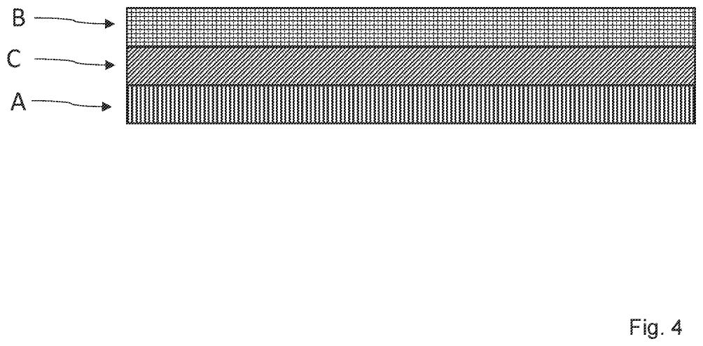

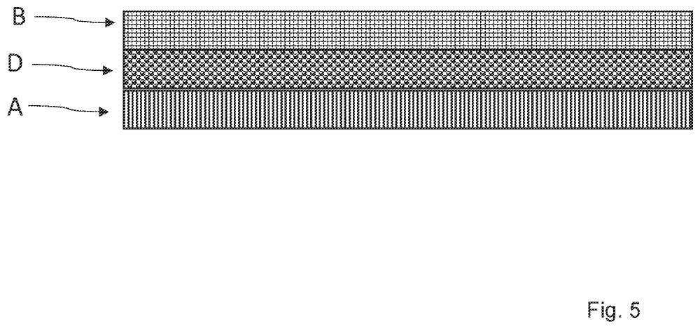

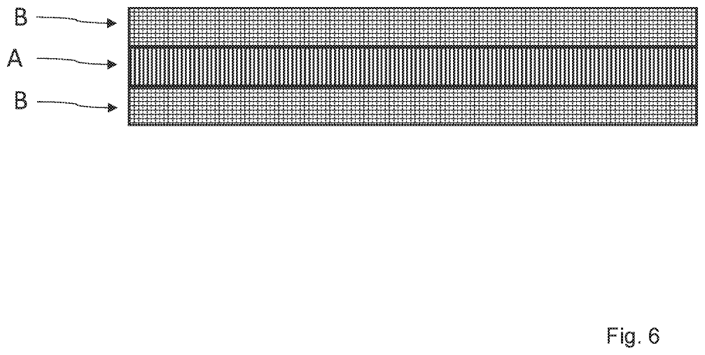

FIGS. 1-8 are cross-section elevation views of portions of various membranes according to embodiments of the present disclosure. The membranes 2 comprises a plurality of layers or components A, B, C, D. As shown and described herein, membranes of the present disclosure contemplate various orderings and arrangements of components. As shown in FIG. 1, for example, one embodiment of the present disclosure contemplates and provides a membrane with a layer-arrangement that can be described as A-B-C-D. As used herein with respect to at least FIGS. 1-8, layer A comprises an immunoisolation membrane with pore diameter in the range of approximately 0.1-5.0 microns and preferably of about 0.4-1.0 microns. The layer A is contemplated as being between 5 and 100 microns in thickness (vertical direction as shown in FIG. 1), and is preferably of about 10-40 microns in thickness. Layer B is contemplated as comprising a vascularization layer with a thickness of between approximately 1 and 100 microns and more preferably of about 5-40 microns. The vascularization layer B comprises pores with pore sizes (diameter) of between 1 and 50 microns, and preferably of about 5 to 20 microns. Layer C is contemplated as comprising a non-woven mesh layer (e.g. a non-woven polyester mesh) with strands of between approximately 5 and 50 microns and preferably of about 10 and 30 microns. Layer D is contemplated as comprising a woven mesh layer (e.g. a woven polyester mesh) with pore sizes of about 20 and 500 microns and strands of approximately 20 and 400 microns in diameter.

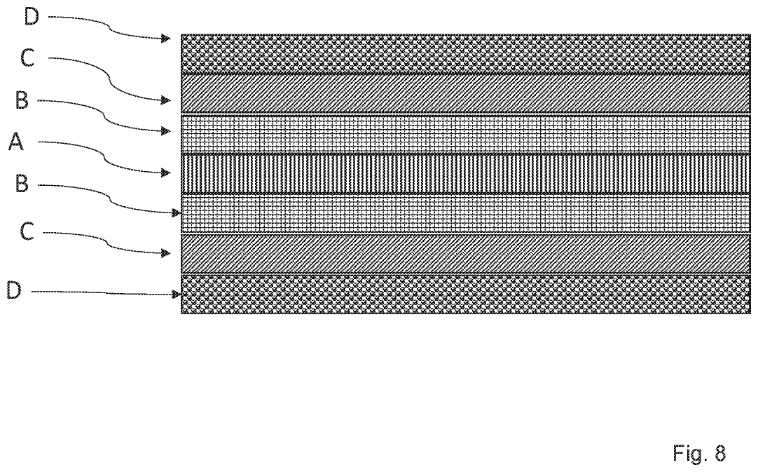

Embodiments of the present disclosure contemplate various combinations and orders of layers A, B, C, D as shown in FIGS. 1-8, for example. It is also contemplated that certain layers may be duplicated or provided more than once in an arrangement. For example, the membrane of FIG. 8 comprises a plurality of non-woven mesh layers C, a plurality of woven mesh layers D, and a plurality of vascularization layers B.

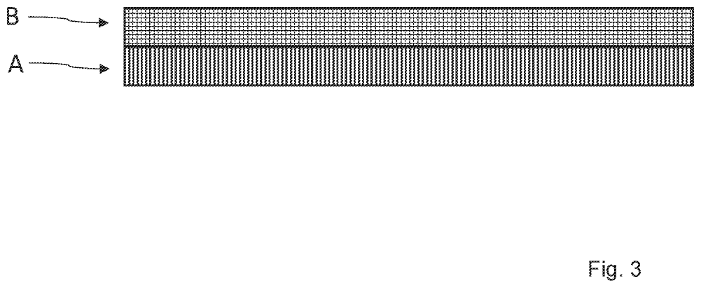

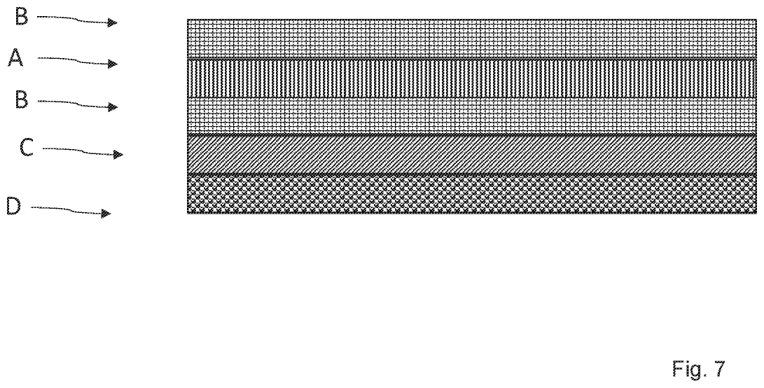

The various membranes contemplated by embodiments of the present disclosure are shown in FIGS. 1-8, for example. The references to different membrane layers A, B, C, D are to be understood to be consistent throughout the Figures and embodiments. For example, the vascularization layer B of FIG. 1 is contemplated as being substantially the same layer B as shown in FIGS. 3 and 7, for example.

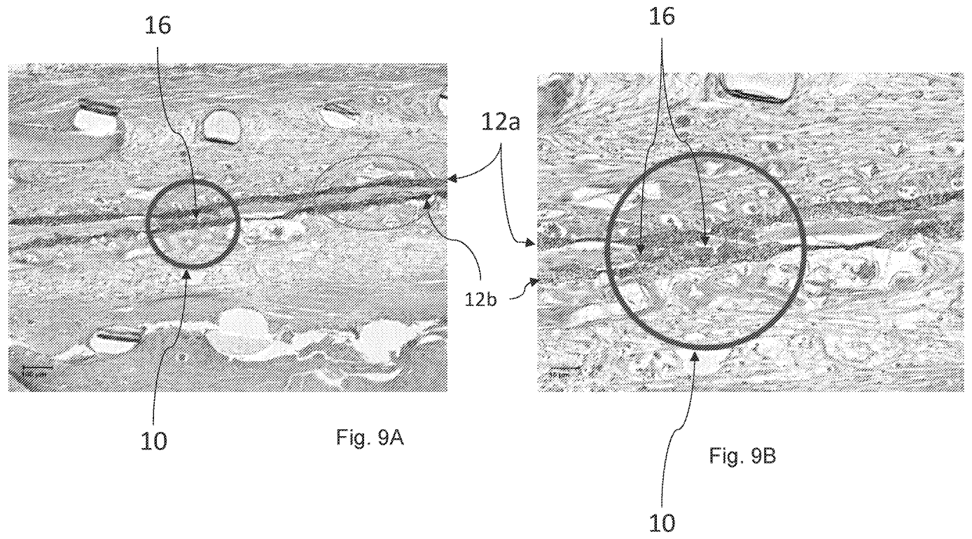

FIG. 9A is a histological view of an implanted membrane device and related tissue. FIG. 9B is a detailed view of FIG. 9A. As shown, in implanted device is shown that comprises first 12a and second 12b vascularization membranes. The vascularization membranes are contemplated as comprising membranes or membrane layers including, for example, those shown and described as element B in FIGS. 1-8. A space or distance between the membranes 12a, 12b is contemplating as being variable along the length of the device, for example. In various embodiments, it is contemplated that a spacing or distance between the features 12a, 12b comprises a distance of between approximately 10 and 200 microns, and preferably of between about 20 and 50 microns. In some embodiments, the distance is approximately 35 microns. The thickness of the vascularization membranes 12a, 12b is contemplated as being between approximately 5 and 50 microns. One of ordinary skill in the art will recognize that a distance between the membranes 12a, 12b will vary based on the position and the thickness of the membranes 12a, 12b. Applicant has discovered that spacing between membranes including but not limited to the vascularization membranes shown in FIGS. 9A-9B can impact the growth and prosperity of vasculature 16 that is formed between layers or features.

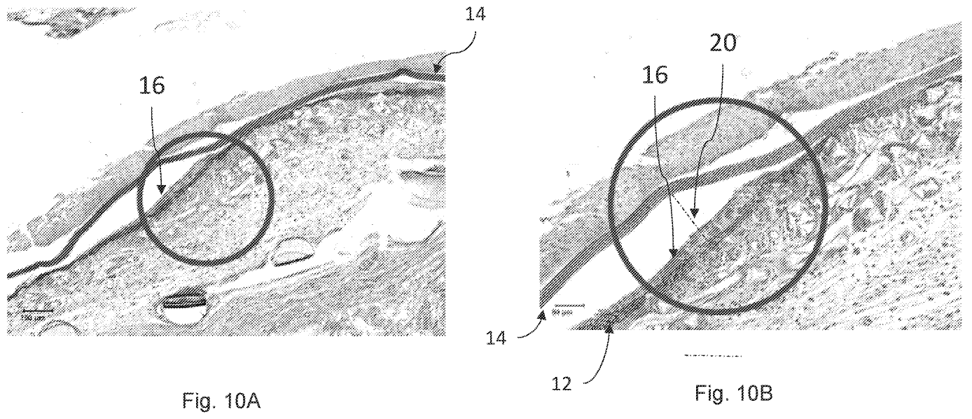

As shown in FIG. 10B, a spacing between the vascularization membrane 12 the immunoisolation membrane 14 is contemplated as being variable. The spacing is contemplated as being as small as 1-10 microns in certain regions and as large as 100-200 microns in other regions. As shown in FIG. 10B, a spacing 20 at a certain portion of the device comprises a spacing of between approximately 50 and 150 microns and preferably of about 100 microns. Areas of preferred spacing as shown in FIG. 10B have been shown by Applicant to provide enhanced vascularization as shown by the presence of in-grown vasculature 16. Embodiments of the present disclosure contemplate various ranges in spacing between components including immunoisolation membranes and vascularization membranes. These ranges of spacing are contemplated as varying between 0 microns and hundreds of microns. In some embodiments it is contemplated that a preferred range of spacing is ideal for promoting vasculature. For example, spacing between adjacent layers or components of approximately 10 microns or less may inhibit desired vascularization. Similarly, excessively large gaps or spacing may inhibit vascularization.

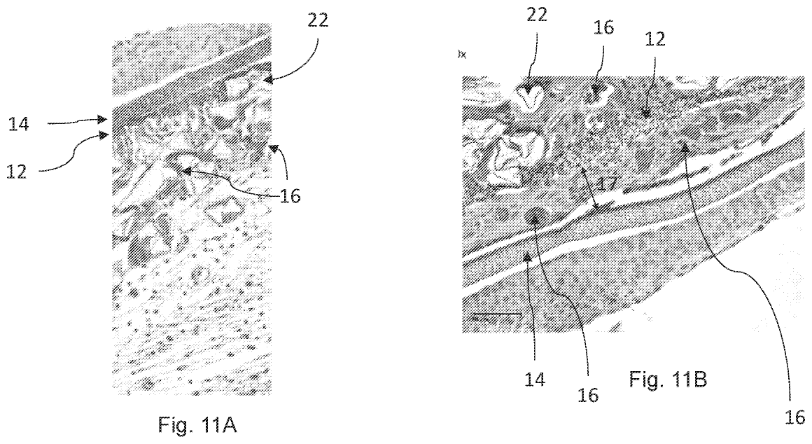

FIGS. 11A-11B provide cross-sectional views of a device according to an embodiment of the present disclosure that comprises a vascularization membrane 12 and an immunoisolation membrane 14. FIG. 11A depicts an embodiment wherein a spacing between the vascularization membrane 12 and an immunoisolation membrane 14 is small. In some embodiments, it is contemplated that the separation between adjacent components (e.g. the vascularization membrane 12 and an immunoisolation membrane 14) is between 0 and 5 microns. Such embodiments with limited or no separation and/or where the components are directly adhered to one another or in direct contact along at least a portion of the device are contemplated. The embodiment of FIG. 11A, for example, enables formation of vasculature 16 within the device. The vasculature 16 in such embodiments have been observed to form at a distance away from the membranes 12, 14. Mesh fibers 22 are provided for reference and are contemplated as providing structural support to the device.

FIG. 11B depicts another embodiment of the present disclosure and wherein a separation or distance is provided between membrane layers. Vasculature 16 ingrowth in a region wherein spacing between the vascularization membrane 12 and an immunoisolation membrane 14 is between approximately 50 and 150 microns, and preferably of about 100 microns. Mesh fibers 22 are also depicted and are provided for structural support. As shown in FIG. 11B, the Applicant has observed increased vascularization 16 density associated with a separation or spacing apart of membrane layers including, for example, a vascularization membrane 12 and an immunoisolation membrane 14. A gap 17 is contemplated as varying in magnitude along a length of the device. In various embodiments, it is contemplated that this gap comprises a distance of between approximately 5 and 250 microns, and more preferably of about 25 to 100 microns. Improved vascularization is provided in the area between the membranes 14, 16 and is also observed at other locations proximal to the membranes.

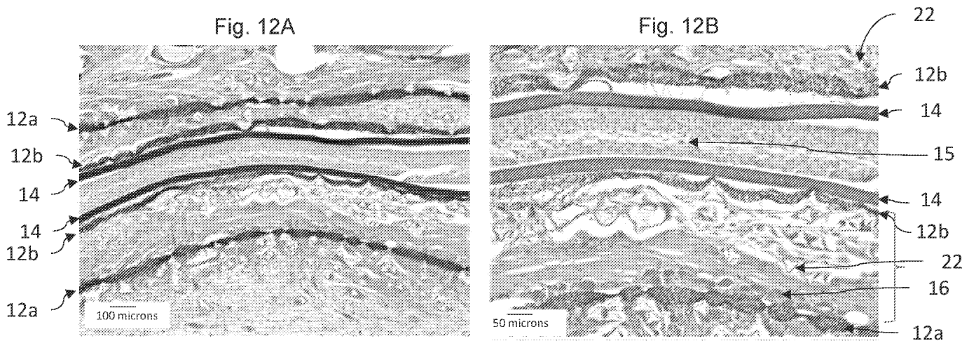

FIGS. 12A-12B provide cross-sectional histological views of a device according to an embodiment of the present disclosure. FIG. 12B is a detailed view of FIG. 12A and FIG. 12D is a detailed view of FIG. 12C. As shown, the device comprises a plurality of membranes including a first vascularization membrane 12a and a second vascularization membrane 12b. An immunoisolation membrane 14 is provided and houses a central volume or lumen 15. Variable spacing is provided between each depicted membrane with the scale(s) shown in microns in FIGS. 12A-12D. As shown in FIG. 12B, for example, vascularization 16 is formed between the first and second vascularization membranes 12a, 12b. Housed cells or therapeutic agents stored within the lumen 15 which is at least partially defined by the immunoisolation membrane 14. The cell(s) or agent(s) are operable to be retained within the immunoisolation membrane and allow diffusion of molecules to the vasculature 16 (for example). One or more ports, gas channels, and/or similar features are contemplated but the lumen is otherwise substantially enclosed.

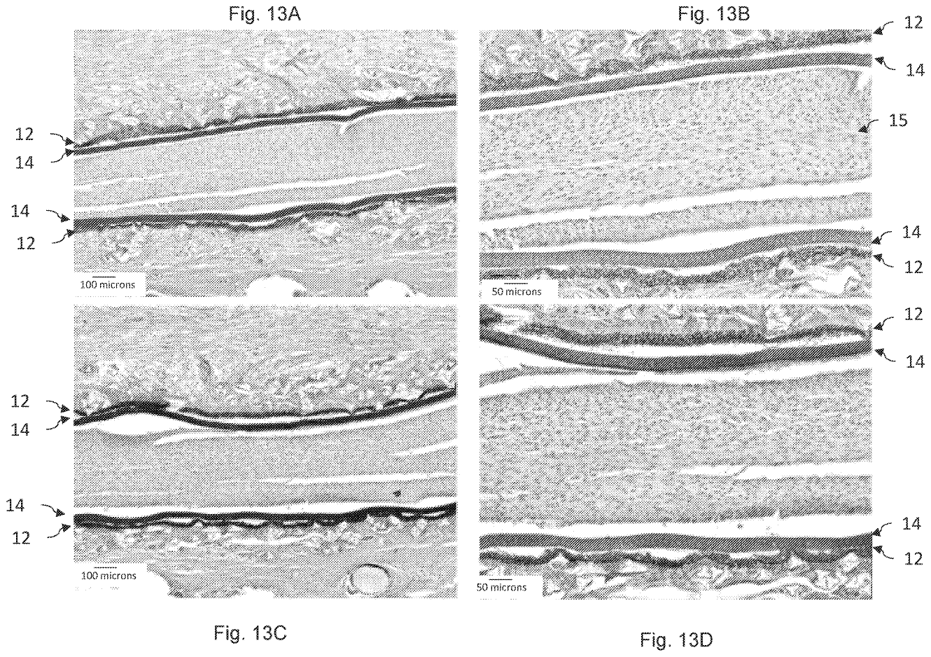

FIGS. 13A-13D are cross-sectional histological views of a device according to an embodiment of the present disclosure. As shown, the device comprises a vascularization membrane 12, an immunoisolation membrane and a space or lumen provided between the immunoisolation membrane. Although not shown by the cross-sectional view of FIGS. 13A-13D, the membranes substantially surround and contain the lumen 15 and are closed at the ends (not shown). One or more ports, gas channels, and/or similar features are contemplated being connected to the lumen but the lumen is otherwise substantially enclosed.

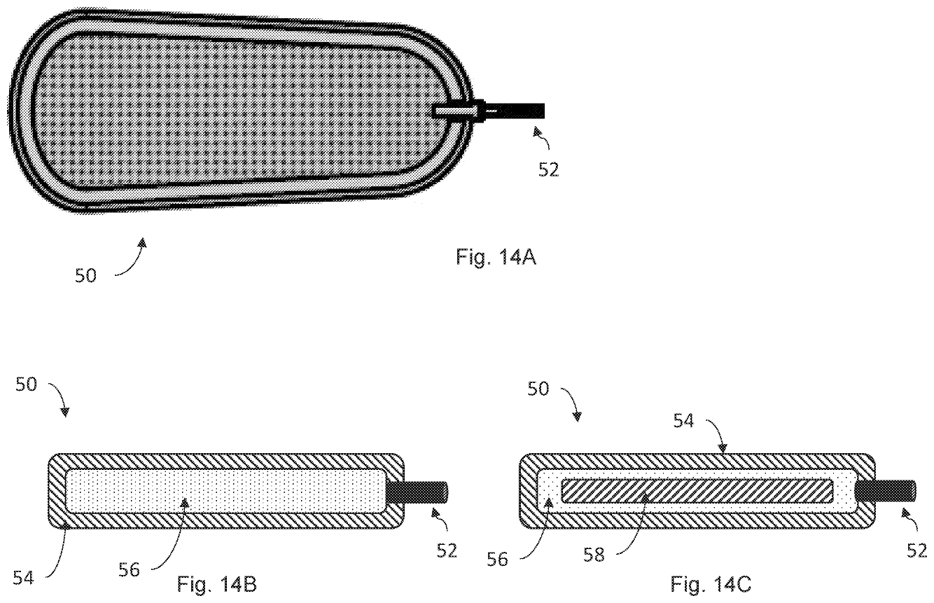

FIG. 14A is a top plan view of a therapeutic agent delivery device 50 according to an embodiment of the present disclosure. As shown, the device 50 comprises a device that is operable to be implanted in a patient and is further operable to house and/or deliver an agent (e.g. insulin). The device 50 is contemplated as comprising a maximized vascularized surface area (VSA) to volume ratio. A conduit or delivery channel 52 is in communication with the device 50. The conduit is contemplated as comprising a fluid delivery line for insulin, for example. However, the conduit 52 may also comprise a gas delivery or cell delivery channel.

FIG. 14B is a cross-sectional elevation view of a device 50 according to an embodiment of the present disclosure. The device 50 comprises a lumen 56. The lumen is contemplated as comprising an internal volume of between approximately 1 and 50 microliters. The lumen is operable to receive at least one of a therapeutic agent (e.g. insulin) and cells for producing a therapeutic effect. The lumen is at least partially defined by the outer membrane portion 54 of the device. The outer membrane portion 54 is contemplated as comprising a vascularizing membrane. Additional features including, for example, an immunoisolation membrane are further contemplated as being provided in the outer membrane portion 54. As will be recognized by one of ordinary skill in the art, the device of FIG. 14B comprises an outer surface with a surface area and the lumen comprises a useful internal volume. A ratio of device surface area to lumen volume for the embodiment of FIG. 14B is referred to herein as X. FIG. 14C is a is a cross-sectional elevation view of a device 50 according to another embodiment of the present disclosure. The device 50 of FIG. 14C comprises a lumen 56. The lumen is contemplated as comprising an internal volume of between approximately 1 and 50 microliters. The lumen is operable to receive at least one of a therapeutic agent (e.g. insulin) and cells for producing a therapeutic effect. The lumen is at least partially defined by the outer membrane portion 54 of the device. The outer membrane portion 54 is contemplated as comprising a vascularizing membrane. Additional features including, for example, an immunoisolation membrane are further contemplated as being provided in the outer membrane portion 54. The device of FIG. 14C further comprises an inert filler 58 to modify the useful or available inner volume of the lumen. The filler 58 is contemplated as comprising silicone or a similar material to occlude or occupy a portion of the lumen and reduce the volume that is available to receive a therapeutic agent (insulin, for example). Accordingly, the device of FIG. 14C comprises a surface area and an internal and a ratio of the surface area to the useful internal volume of the device Y, and wherein Y is greater than the aforementioned ratio X of the device of FIG. 14B. Devices of the present disclosure including, for example, the device of FIG. 14C comprises a preferred ratio wherein the effective volume is reduced to enable a safe and preferred amount of insulin (for example) to be delivered to the device without reducing the surface area that is available for vascularization.

One or more filler materials are contemplated as being provided to decrease the usable or active internal volume of the device. In some embodiments it is contemplated that the internal volume of the lumen 54 comprises a volume of approximately 25 microliters and an inert filler is provided that occupies between 5 and 20 microliters of that volume effectively reducing the usable volume of the lumen 54 to between approximately 5 and 20 microliters and without reducing the overall size of the device or the external surface area. Such a device is contemplated as comprising a 1.5 square centimeter device external surface (FIG. 14A, for example) that is operable to be vascularized.

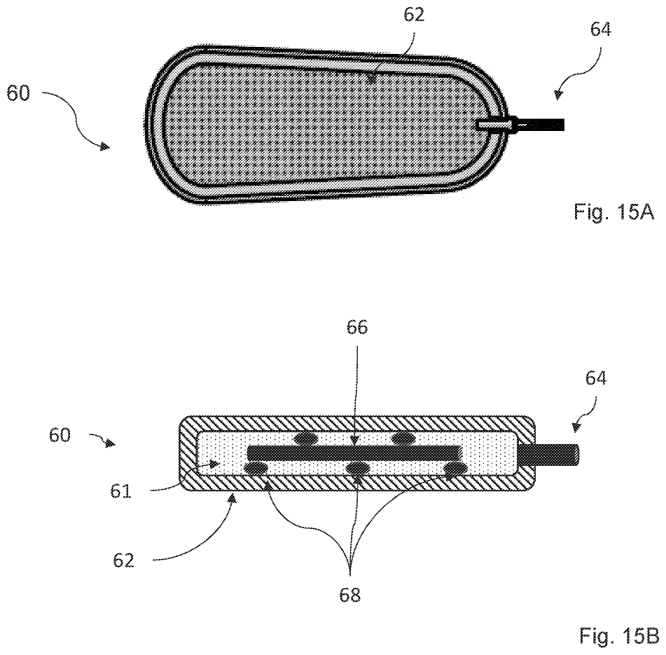

FIG. 15A is a top plan view of a delivery device 60 according to an embodiment of the present disclosure. As shown, the device comprises an implantable delivery device that is operable for delivering insulin (for example) to a patient. In some embodiments, the device 60 comprises a 3D printed silicone member with ports 62 or apertures for delivery and distribution of an agent (e.g. insulin). A delivery port 64 is provided and is contemplated as conveying agents, fluids, etc. to the device 60 from various sources. FIG. 15B is a cross-sectional elevation view of the device 60 of FIG. 15A. As shown, one or more filler materials are provided within the device such that the total interior volume of the device is reduced without reducing an external surface area of the device that is available to promote vascularization. Filler 66 is contemplated as comprising various insert materials and may be glued or spot welded 68 within the chamber to avoid unwanted expansion of the internal volume, particularly when under pressure. In some embodiments, a catheter and/or tubing extends within the internal volume of the device to occupy at least some of the internal volume of the device. The catheter is contemplated as comprising an extension and/or is in fluid communication with the delivery port 64. The filler material 66 is contemplate as comprising various configurations including, for example, serpentine configurations to occupy a desired amount of space within the device.

Implantable devices of the present disclosure including but not limited to the device of FIGS. 15A-15B are contemplated as being operable to deliver at least one of anti-inflammatory agents and immunosuppressive drugs (e.g. cyclosporin) to improve vascularization and minimize inflammation. In some embodiments, a second fluid channel is provided that is separate from the first fluid channel for drug delivery. The second fluid channel is operable to convey at least one of anti-inflammatory agents and immunosuppressive drugs to a region of the device where vascularization is expected and/or encouraged to grow. The second channel and related fluid delivery is operable to reduce or minimize inflammation around the device. In some embodiments, a channel is provided that is operable to delivering a cooling agent (e.g. reduced temperature fluid(s) such as air or inert gas) to reduce inflammation around the device.

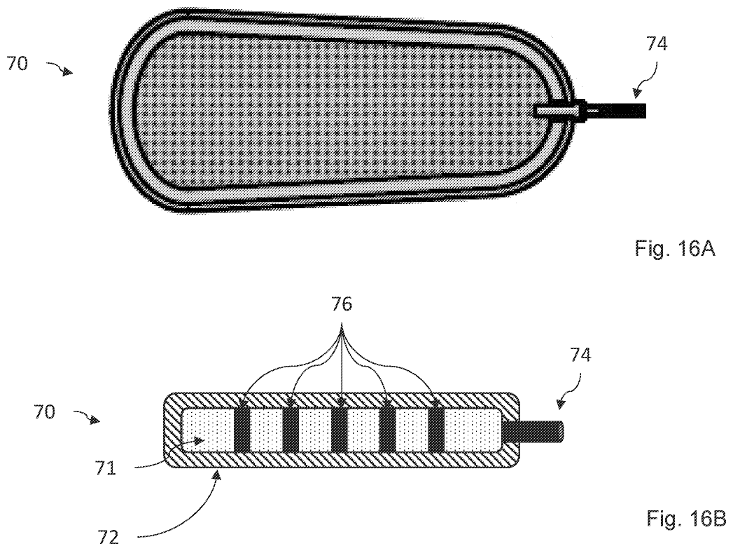

FIG. 16A is a top plan view of an implantable device 70 according to an embodiment of the present disclosure. As shown, the device 70 comprises an implantable device or wafer that is operable to be implanted subcutaneously. FIG. 16B is a cross-sectional elevation view of the device of FIG. 16A. As shown, the device 70 comprises an internal lumen 71. A plurality of posts 76 are provided. The posts are contemplated as comprising one or more inert materials including, for example, silicone. The posts are operable to at least one of occupy a space or volume within the lumen to reduce the available lumen volume that can receive fluid (insulin, for example) and to provide structural support to the device. The structural support is provided at least in part by the connection of the posts to opposing sides of the outer membrane layer 72 and wherein the posts prevent or reduce expansion of the lumen (when fluid or pressure is provided to the lumen, for example) and unwanted compression of the lumen (when external forces are applied, for example). Although five posts 76 are shown in FIG. 16B, no limitation is provided with respect to the number, arrangement or distribution of posts is provided. The present disclosure contemplates devices with one or more posts 76 in variety of arrangements. Additionally, posts or supports are not limited to the vertical arrangement shown in FIG. 16B. For example, wave-like inserts, truss members, spherical inserts, and other arrangements are contemplated.

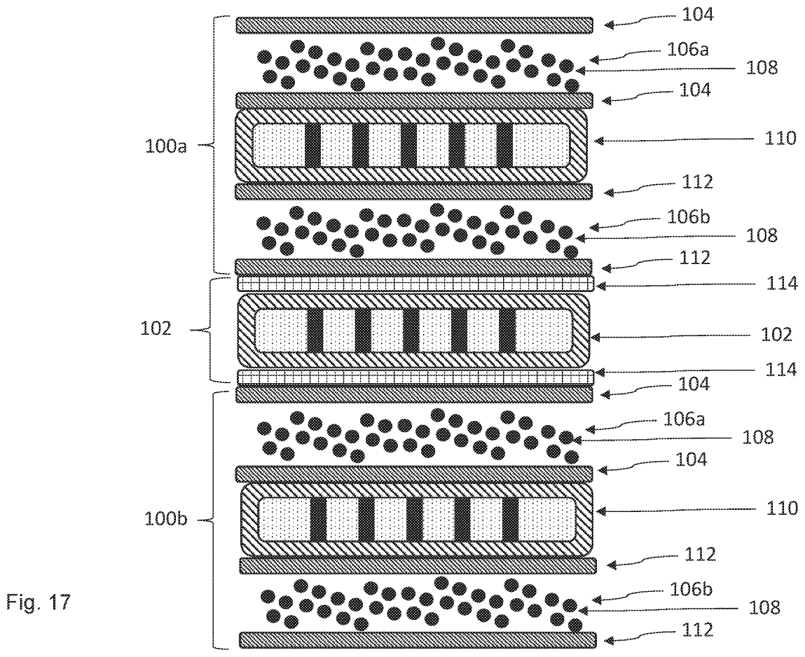

FIG. 17 is a cross-sectional view of a device according to an embodiment of the present disclosure. The device generally comprises three regions or stacked components including a first non-immunoisolating encapsulation device, a second non-immunoisolating encapsulation device, and a vascularized oxygen chamber, the details of which are described in detail below. FIG. 17 shows an arrangement and ordering of layers and it should be recognized that the figure is not to scale and the length and width of the device are not shown in their entireties.

The encapsulation devices 100a, 100b of FIG. 17 each comprise a vascularization membrane 104 at least partially defining a lumen 106. The lumen 106a is depicted as housing cells 108. The lumen is also operable to receive a therapeutic agent (e.g. insulin). A wafer 110 is provided that is contemplated as comprising a silicone wafer operable to act as a spacer, provide oxygen to the system, and/or enable vascularization. The wafer 110 is contemplated as comprising various structures including but not limited to the wafers shown and described herein (including that of FIG. 16B, for example). A second vascularization membrane 112 is provided that surrounds and at least partially defines a lumen 106b. Collectively, the two membranes 104, 112 and the wafter 110 comprise a first non-immunoisolating encapsulation device 100a.

A vascularized oxygen chamber 102 is provided between the non-immunoisolating encapsulation devices 100a, 100b. The vascularized oxygen chamber 102 comprises one or more vascularization membranes or layers 114 that surround a wafer or implant that is operable to receive fluid (e.g. oxygen or oxygenated gas) and provide oxygen to at the adjacent cell encapsulation devices.

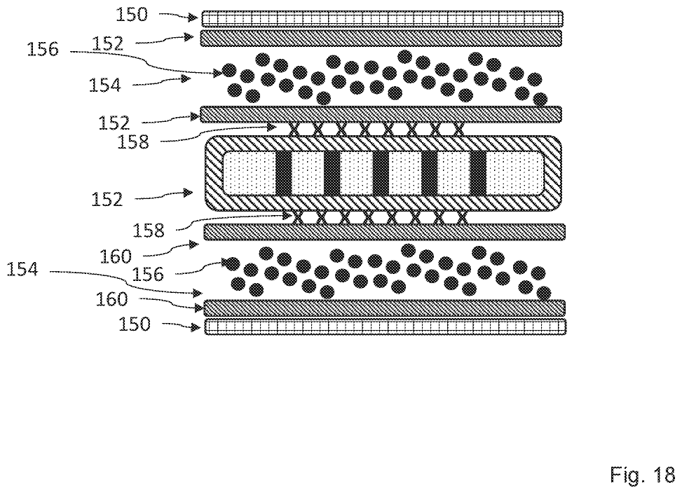

FIG. 18 is a cross-sectional view of a device according to an embodiment of the present disclosure. The device generally comprises multiple regions or stacked components including a first non-immunoisolating encapsulation device, a second non-immunoisolating encapsulation device, and a vascularized oxygen chamber, the details of which are described in detail below. FIG. 18 shows an arrangement and ordering of layers and it should be recognized that the figure is not to scale and the length and width of the device are not shown in their entireties.

As shown in FIG. 18, the device comprises a nonimmunoisolating device comprising a woven mesh layer 150. First and second cell encapsulation devices comprising a vascularization membrane 152, 160 are provided that are operable to house cells 156 in a lumen 154. An oxygen delivery device 152 is provided that comprises a wafer or insert operable to deliver oxygen (for example) and enhance vascularization of the device. The wafer 152 comprises a preferably insert device comprising silicone, for example, and comprises a void space operable to deliver fluid including but not limited to oxygenated gas to the device. As shown in FIG. 18, the wafer 152 is secured within the system by at least one of sutures and welds 158. In preferred embodiments, the wafer 152 is secured to adjacent encapsulation devices by spot welds. The welds 158 are also operable to provide a distance or spacing between the wafer 152 and the adjacent vascularization membranes.

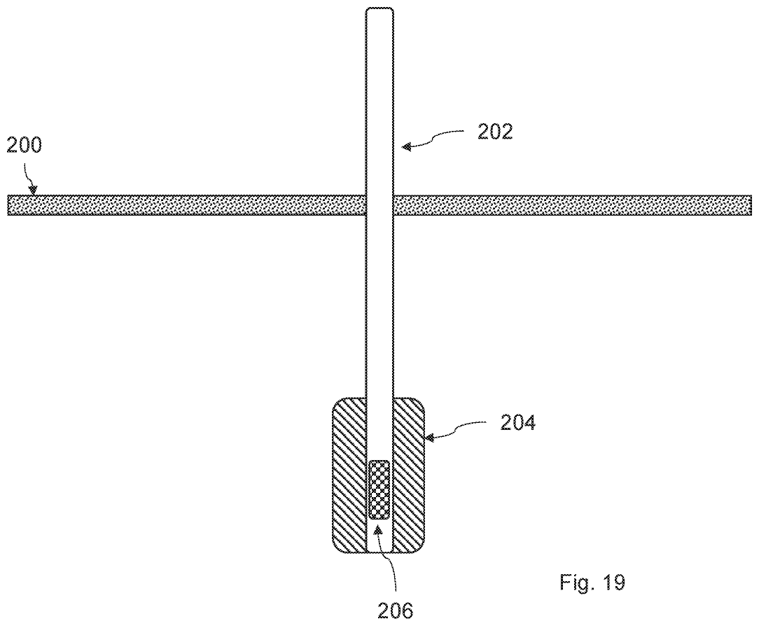

FIG. 19 depicts an embodiment of the present disclosure comprising an implantable chamber 204 at a subcutaneous location with the skin 200 of a patient provided for reference. The chamber 204 preferably comprises a vascularized chamber that is operable to receive at least one of a sensor, cells, and therapeutic agent(s). A conduit 202 is provided that is operable to provide an implantation pathway. The conduit 202 is contemplated as comprising various features including, for example, a vascularized catheter as shown and described herein, a selectively connectable conduit, and similar features. FIG. 19 depicts a cartridge 206 provided in the implantable device and wherein the cartridge 206 is implantable via the conduit 202. The cartridge 206 is contemplated as comprising at least one of a sensor, cells, and a therapeutic agent.

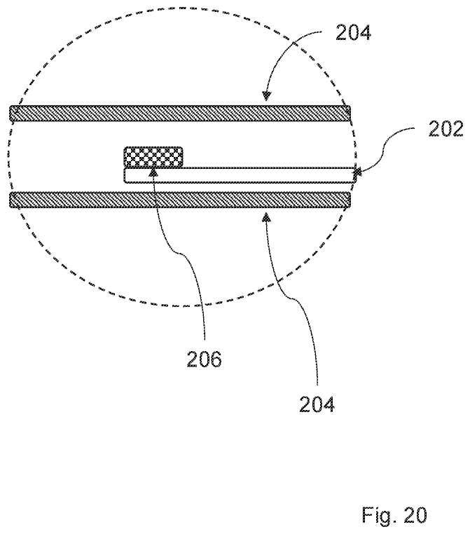

FIG. 20 is a cross-sectional elevation view of the embodiment of FIG. 19. As shown, a cartridge 206 is provided within a vascularized chamber comprising at least one membrane or vascularized layer 204. The cartridge is contemplated as being implantable and extractable via the conduit 202.

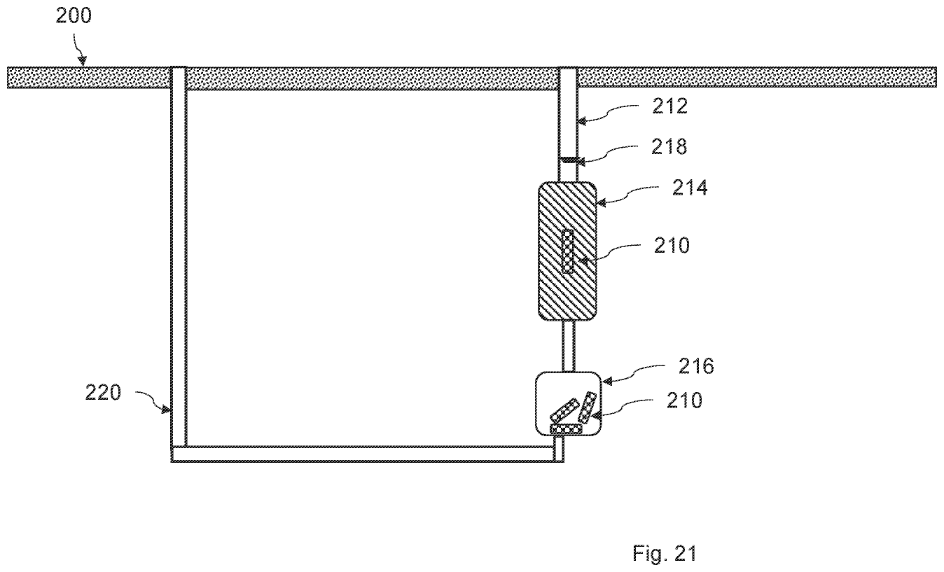

FIG. 21 is an illustration of a system according to an embodiment of the present disclosure. The system comprises an implantable system that is operable to receive one or more sensors 210. As shown, the system comprises a subcutaneous conduit or channel 212 that extends beneath the skin 200. A chamber 214 is preferably provided beneath the skin and the chamber 214 is operable to receive at least one sensor 210 via the conduit 212. Sensors contemplated for use with the present disclosure (including but not limited to the embodiment of FIG. 21) and to be provided within the system are contemplated as comprising various different types of sensors for various different purposes. The sensors, for example, are contemplated as comprising electrochemical sensors, NMR sensors, optical sensors and similar devices and are operable to sense, detect or evaluate one or more of oxygen levels, glucose levels, temperature, etc. Implantable sensors as contemplated for use with the depicted embodiment are known to comprise a finite lifespan that is generally shorter than the lifespan of an implantable system. Accordingly, sensors will require removal and/or replacement during the life of the system in order to continue to monitor conditions associated with the system.

The system of FIG. 21 comprises a chamber 214 for receiving a working sensor 210. A sensor collection feature or repository 216 is provided in series with the chamber 214. The repository 216 is operable to receive spent or used sensors that are no longer functional. Sensors 210 are collected in the repository in a manner that reduces the need or frequency at which surgical procedures must be performed to remove the sensors 210. Existing systems and methods contemplate some level of invasive surgery to remove and/or replace a sensor upon sensor expiration. The present disclosure and the embodiment of FIG. 21 provide for a significantly reduced need for such procedures wherein sensors 210 may be “pushed” or forced into the chamber or repository 216 and housed therein until a removal operation is performed. Such features and systems reduce the need for surgical intervention where, for example, removal procedures are required on the order of months of years as opposed to weeks.

FIG. 21 depicts one physical arrangement of components wherein the repository 216 is provided below and at a greater subcutaneous depth than the chamber 214. It will be recognized, however, that the present disclosure is not limited to such an arrangement. It is contemplated, for example, that the repository may be provided at the same depth as the chamber 214. The repository may be provided at various subcutaneous depths and, in some embodiments, is provided proximal to the skin 200 to facilitate sensor removal operations.

The embodiment of FIG. 21 further comprises a sensor activation feature 218. In some embodiments, it is contemplated that new or unused sensors initially comprise a film or seal that must be removed in order to activate the sensor. Embodiments of FIG. 21 contemplate a structural member that scrapes or removes the film prior to the sensor entering the chamber 214 and wherein the sensor is automatically activated as it is transmitted through the channel 212. In alternative embodiments, a chemical means is provided in addition to or in lieu of the activation feature 218. For example, in some embodiments, the sensors are initially provided with an alginate coating that is to be removed prior to use. Embodiments and systems of the present disclosure contemplate the provision of an agent (e.g. sodium citrate) to dissolve and remove the alginate barrier and activate the sensor(s).

The embodiment of FIG. 21 contemplates and depicts a removal conduit 220 for sensors. As shown, the removal conduit extends from the repository 216 to the skin 200 or a location just below the skin. While certain embodiments of the present disclosure are devoid of the depicted removal conduit and rely solely on surgical or manual removal of the sensors directly from the repository 216, further embodiments comprise a pathway 220 for transmitting used or expired sensors from the body.

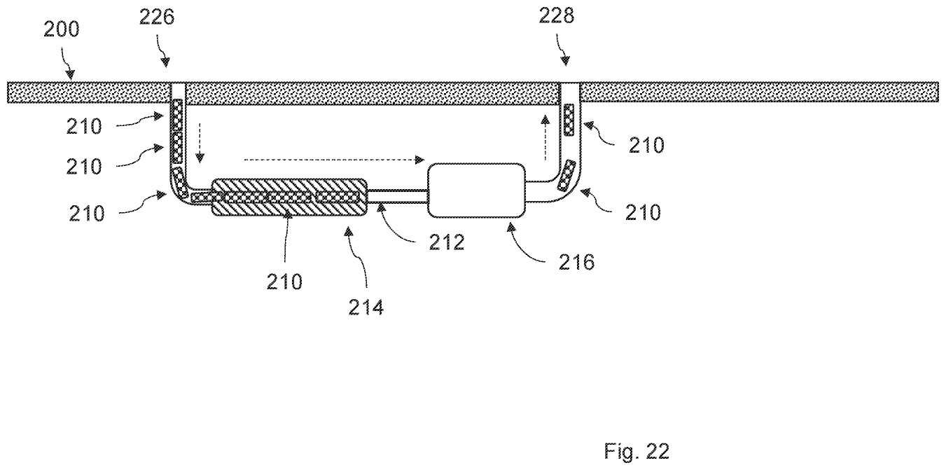

FIG. 22 illustrates an implantable system according to an embodiment of the present disclosure. As shown, the system comprises a pathway for a series of sensors 210. The sensors 210 are contemplated as being provided in series and, in some embodiments, are provided in an adjacent or stacked arrangement and are provided to a chamber 214 as needed. Accordingly, a force applied to a sensor 210 is operable to be transmitted to an adjacent sensor 210. Sensors are moved into and out of a chamber 214 in series and spent or expired sensors are moved out of the chamber and provided to a repository 216 and/or removed from the patient.

Embodiment of the present disclosure contemplate that an activated sensor is provided to the chamber 214 and is useful for approximately 3-6 months or longer. Once used and/or expired, the sensor is displaced from the chamber by new, adjacent sensor(s).

Although the embodiments of the present disclosure depict sensors provided to a subcutaneous system, it is contemplated that systems and features of the present disclosure contemplate cartridge, cells, therapeutic agents, and similar features in place of various sensors as shown in FIGS. 21-22 (for example).

The examples set forth above are provided to give those of ordinary skill in the art a complete disclosure and description of how to make and use the embodiments of the methods for delivery of a biomolecule of interest and are not intended to limit the scope of what the inventors regard as the scope of the disclosure. Modifications of the above-described modes for carrying out the disclosure can be used by persons of skill in the art and are intended to be within the scope of the following claims.

It is to be understood that the disclosure is not limited to particular methods or systems, which can, of course, vary. It is also to be understood that the terminology used herein is for the purpose of describing particular embodiments only, and is not intended to be limiting.

A number of embodiments of the disclosure have been described. Nevertheless, it will be understood that various modifications may be made without departing from the spirit and scope of the present disclosure. Accordingly, other embodiments are within the scope of the following claims.

What is claimed is:

1. A membrane for implantable medical devices, the membrane comprising:
a vascularization membrane operable to enable and/or permit vasculature to grow;
a mesh layer; and
wherein the immunoisolation membrane and the vascularization membrane comprise adjacent and spaced apart layers and wherein a separation is provided between the immunoisolation membrane and the vascularization membrane and the separation is at least approximately 50 microns and wherein vasculature is operable to be provided in the separation.

2. The membrane of claim 1, wherein the vascularization membrane is provided adjacent to an immunoisolation membrane.

3. The membrane of claim 1, wherein the mesh layer comprises a woven mesh layer.

4. The membrane of claim 1, wherein the mesh layer comprises a non-woven mesh layer and wherein the membrane further comprises a woven mesh layer.

5. The membrane of claim 2, wherein the immunoisolation membrane comprises pores having a diameter of between approximately 0.4 and 1.0 microns.

6. The membrane of claim 1, wherein the membrane at least partially defines a lumen for receiving at least one of cells and a therapeutic agent.

7. The membrane of claim 1, wherein the mesh layer comprises polyester.

8. The membrane of claim 1, wherein the vascularization membrane comprises pores and the pore diameters are between approximately 5 and 25 microns.

9. A membrane for implantable medical devices, the membrane comprising:
an immunoisolation layer;
a vascularization layer operable to enable and/or permit vasculature to grow into the vascularization layer;
a non-woven mesh layer;
a woven mesh layer; and
wherein two or more of the layers are provided in a stacked arrangement and secured to one another by at least one of sutures and welds.

10. The membrane of claim 9, wherein the vascularization layer is provided adjacent to the immunoisolation layer, the non-woven mesh layer is provided adjacent to the vascularization layer, and the woven mesh layer is provided adjacent to the woven mesh layer.

11. The membrane of claim 9, wherein the immunoisolation layer comprises a thickness of between approximately 10-40 microns.

12. The membrane of claim 9, wherein the immunoisolation layer comprises pores having a diameter of between approximately 0.4 and 1.0 microns.

13. The membrane of claim 9, wherein the membrane at least partially defines a lumen for receiving at least one of cells and a therapeutic agent.

14. The membrane of claim 9, wherein at least one of the non-woven mesh layer and the woven mesh layer comprise polyester.

15. The membrane of claim 9, wherein the vascularization layer comprises pores and the pore diameters are between approximately 5.0 and 25.0 microns.
